(19) United States
(12) Patent Application Publication
(10) Pub. No.: US 2005/0214332 A1
(43) Pub. Date: Sep. 29, 2005

(54) Skin care composition containing dehydroacetic acid and skin care actives

(75) Inventors: Rosemarie Osborne, Oxford, OH (US); Larry Richard Robinson, Loveland, OH (US); Paul Robert Tanner, Lebanon, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/977,848

(22) Filed: Oct. 29, 2004

Related U.S. Application Data
(60) Provisional application No. 60/516,502, filed on Oct. 31, 2003.

(51) Int. Cl. A61K 7/06; A61K 7/11

(52) U.S. Cl. 424/401; 424/70.13

(57) ABSTRACT

Skin care compositions containing dehydroacetic acid, its isomers, salts, and derivatives thereof; at least two skin care actives selected from sugar amines, vitamin B3 compounds, phytosterols, salicylic acid compounds, hexamidines, dialkanoyl hydroxyproline compounds, flavonoids, n-acyl amino acid compounds, and their derivatives, and combinations thereof; and a dermatologically acceptable carrier for the dehydroacetic acid and the skin care actives. The invention further relates to methods for regulating the condition of mammalian keratinous tissue wherein the methods each comprise the step of topically applying to the keratinous tissue of a mammal needing such treatment, a safe and effective amount of the skin care composition of the invention.

### CROSS REFERENCE TO RELATED APPLICATION(S)

This application claims the benefit of U.S. Provisional Application 60/516,502, filed Oct. 31, 2003.

### TECHNICAL FIELD

The present invention relates to a skin care composition containing dehydroacetic acid, its isomers, salts, or derivatives thereof; skin care actives; and a dermatologically acceptable carrier for the dehydroacetic acid and the skin care actives. Such compositions are useful for regulating mammalian keratinous tissue conditions.

### BACKGROUND OF THE INVENTION

Currently, there are a number of skin care products that are available to consumers, which are directed toward improving the health and physical appearance of keratinous tissues such as the skin, hair, and nails. Mammalian keratinous tissue, particularly human skin, is subject to a variety of insults by both extrinsic and intrinsic factors. Extrinsic factors include ultraviolet radiation, environmental pollution, wind, heat, infrared radiation, low humidity, harsh surfactants, abrasives, etc. Intrinsic factors include chronological aging and other biochemical changes from within the skin. Whether extrinsic or intrinsic, these factors result in visible signs of aging.

The majority of products treating skin conditions caused by these extrinsic and intrinsic factors are directed to delaying, minimizing or even eliminating skin wrinkling. However, there exists a need for cosmetic agents to prevent, retard, and/or treat uneven skin texture. For example skin that has poor skin texture is not smooth tactilly and/or visually and can be characterized by being bumpy, having large pores and uneven appearance. There is a need to provide products to consumers that treat keratinous tissue conditions such as enhancing tactile and visual skin texture and creating a more uniform appearance of skin by improving skin smoothness, improving skin tone, reducing the oiliness of skin, and reducing pore size.

### SUMMARY OF THE INVENTION

Without being limited by theory, it has been found that certain compositions containing a combination of dehydroacetic acid and at least two additional skin care actives can prevent, retard, and/or treat uneven skin texture by acting as an oil control and pore reduction cosmetic agent. Consequently, Applicants have surprisingly found that topical compositions that contain dehydroacetic acid and at least two additional skin care actives including sugar amines, vitamin B3 compounds, phytosterols, salicylic acid compounds, hexamidines, dialkanoyl hydroxyproline compounds, flavonoids, N-acyl amino acid compounds, and their derivatives, and combinations thereof may be used to provide prophylactic as well as therapeutic treatments for keratinous tissue conditions.

Applicants have surprisingly found that topical compositions that contain dehydroacetic acid and at least three additional skin care actives including sugar amines, vitamin B3 compounds, phytosterols, salicylic acid compounds, hexamidines, dialkanoyl hydroxyproline compounds, flavonoids, N-acyl amino acid compounds, retinoids, peptides, water-soluble vitamins, particulate materials, sunscreen actives, anti-cellulite agents, butylated hydroxytoluene and butylated hydroxyanisole, and their derivatives, and combinations thereof may be used to provide prophylactic as well as therapeutic treatments for keratinous tissue conditions.

Applicants have found that such compositions may be useful for preventing, retarding, and/or treating uneven skin texture by regulating oily/shiny appearance, and regulating and/or reducing pore size appearance.

The present invention relates to a skin care composition comprising:
a) dehydroacetic acid, its isomers, salts, and derivatives thereof;
b) a safe and effective amount of at least two skin care actives selected from the group consisting of sugar amines, vitamin B3 compounds, phytosterols, salicylic acid compounds, hexamidines, dialkanoyl hydroxyproline compounds, flavonoids, n-acyl amino acid compounds, and their derivatives, and combinations thereof; and
c) a dermatologically acceptable carrier for the dehydroacetic acid and the skin care actives.

The invention further relates to methods for regulating the condition of mammalian keratinous tissue wherein the methods each comprise the step of topically applying to the keratinous tissue of a mammal needing such treatment, a safe and effective amount of the skin care composition of the invention.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application”, as used herein, means to apply or spread the compositions of the present invention onto the surface of the keratinous tissue.

The term “dermatologically acceptable,” as used herein, means that the compositions or components described are suitable for use in contact with human keratinous tissue without undue toxicity, incompatibility, instability, allergic response, and the like.

The term “safe and effective amount,” as used herein, means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The terms “oily and/or shiny appearance” as used herein mean the glossy look mammalian skin tends to exhibit upon the excretion of oil, sebum, and/or sweat from the respective source gland.

The term “smoothing” and “softening” as used herein means altering the surface of the keratinous tissue such that its tactile feel is improved.

The term “regulating the skin condition” includes prophylactically regulating and/or therapeutically regulating skin condition. As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing), discontinuities in skin. Regulating skin condition involves improving skin appearance and/or feel. As used herein, “regulating skin condition” is intended to include regulation of such signs irrespective of the mechanism of origin.

The compositions of the present invention, including the essential and optional components thereof, are described in detail hereinafter.

### Components

1. Dehydroacetic Acid (DHA)

The composition of this invention comprises dehydroacetic acid, having the structure: 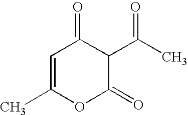
or pharmaceutically acceptable salts, derivatives or tautomers thereof. As used herein, “pharmaceutically acceptable” means that the salts of dehydroacetic acid are suitable for use in contact with the tissues of mammals to which they will be exposed without undue toxicity, incompatibility, instability, irritation, allergic response, and the like. The technical name for dehydroacetic acid is 3-Acetyl-6-methyl-2H-pyran-2,4(3H)-dione and can be commercially purchased from Lonza.

Pharmaceutically acceptable salts include alkali metal salts, such as sodium and potassium; alkaline earth metal salts, such as calcium and magnesium; non-toxic heavy metal salts; ammonium salts; and trialkylammonium salts, such astrimethylammonium and triethylammonium. Sodium, potassium, and ammonium salts of dehydroacetic acid are preferred. Highly preferred is sodium dehydroacetate which can be purchased from Tri-K, as Tristat SDHA. Derivatives of dehydroacetic acid incude, but are not limited to, any compounds wherein the CH₃ groups are individually or in combination replaced by amides, esters, amino groups, alkyls, and alcohol esters. Tautomers of dehydroacetic acid are the isomers of dehydroacetic acid which can change into one another with great ease so that they ordinarily exist in equilibrium. Thus, tautomers of dehydroacetic acid can be described as having the chemical formula C₈H₈O₄ and generally having the structure above.

The compositions of the present invention comprise from about 0.001% to about 25% by weight of the composition, preferably from about 0.01% to about 10%, more preferably from about 0.05% to about 5%, and even more preferably from about 0.1% to about 1% of dehydroacetic acid or pharmaceutically acceptable salts, derivative or tautomers thereof.

2. Skin Care Actives

The present invention includes at least two different skin care actives that are selected from the group consisting of sugar amines, vitamin B3 compounds, phytosterols, salicylic acid compounds, hexamidines, dialkanoyl hydroxyproline compounds, flavonoids, and N-acyl amino acid compounds, their derivatives, and combinations thereof. The present invention also includes at least three different skin care actives that are selected from the group consisting of sugar amines, vitamin B3 compounds, phytosterols, salicylic acid compounds, hexamidines, dialkanoyl hydroxyproline compounds, flavonoids, N-acyl amino acid compounds, retinoids, peptides, water-soluble vitamins, particulate materials, sunscreen actives, anti-cellulite agents, butylated hydroxytoluene and butylated hydroxyanisole, their derivatives, and combinations thereof.

1. Sugar Amines (Amino Sugars)

The compositions of the present invention may include a safe and effective amount of a sugar amine, which is also known as amino sugars. The sugar amine compounds useful in the present invention are described in PCT Publication WO 02/076423 and U.S. Pat. No. 6,159,485.

Preferably, the composition contains from about 0.01% to about 15%, more preferably from about 0.1% to about 10%, and even more preferably from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

2. Vitamin B₃ Compounds

The compositions of the present invention may include a safe and effective amount of a vitamin B₃ compound. Vitamin B₃ compounds are particularly useful for regulating skin conditions as described in U.S. Pat. No. 5,939,082. Preferably, the composition contains from about 0.001% to about 50%, more preferably from about 0.01% to about 20%, even more preferably from about 0.05% to about 10%, and still more preferably from about 0.1% to about 7%, even more preferably from about 0.5% to about 5%, by weight of the composition, of the vitamin B₃ compound.

As used herein, “vitamin B₃ compound” means a compound having the formula: 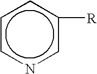
wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate, myristyl nicotinate).

Examples of suitable vitamin B₃ compounds are well known in the art and are commercially available from a number of sources (e.g., the Sigma Chemical Company, ICN Biomedicals, Inc., and Aldrich Chemical Company).

3. Phytosterol

The topical compositions of the present invention may comprise a safe and effective amount of one or more phytosterols selected from the group consisting of β-sitosterol, campesterol, brassicasterol, Δ5-avennasterol, lupenol, α-spinasterol, stigmasterol, their derivatives, analogs, and combinations thereof. More preferably, the phytosterol is selected from the group consisting of β-sitosterol, campesterol, brassicasterol, stigmasterol, their derivatives, and combinations thereof. More preferably, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

In the compositions of the present invention, the phytosterol preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2% by weight of the composition.

4. Salicylic Acid Compound

The topical compositions of the present invention may comprise a safe and effective amount of a salicylic acid compound, its esters, its salts, or combinations thereof. In the compositions of the present invention, the salicylic acid compound preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2%, by weight of the composition, of salicylic acid.

5. Hexamidine

The compositions of the present invention may include hexamidine compounds. The hexamidine compounds useful in the present invention correspond to those of the following chemical structure: 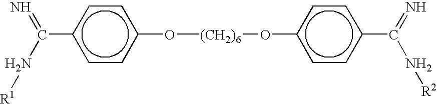
wherein R¹ and R² are organic acids (e.g., sulfonic acids, etc.).

In the composition of the present invention, the hexamidine preferably comprises from about 0.0001% to about 25%, more preferably from about 0.001% to about 10%, more preferably from about 0.01% to about 5%, and even more preferably from about 0.02% to about 2.5% by weight of the composition.

The topical compositions of the present invention optionally include a safe and effective amount of one or more of hexamidine compounds, its salts, and its derivatives. As used herein, hexamidine derivatives include any isomers and tautomers of hexamidine compounds including but not limited to organic acids and mineral acids, for example sulfonic acid, carboxylic acid, etc. Preferably, the hexamidine compounds include hexamidine diisethionate, commercially available as Eleastab® HP100 from Laboratoires Serobiologiques.

6. Dialkanoyl Hydroxyproline Compounds

The compositions of the present invention may comprise a safe and effective amount of one or more dialkanoyl hydroxyproline compounds and their salts and derivatives. The dialkanoyl hydroxyproline compounds of the present invention correspond to those of the following chemical structure: 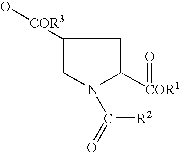
wherein R¹ is H, X, C₁-C₂₀ straight or branched alkyl,
X is metals (Na, K, Li, Mg, Ca) or amines (DEA, TEA);
R² is C₁-C₂₀ straight or branched alkyl;
R³ is C₁-C₂₀ straight or branched alkyl.

In the composition of the present invention, the dialkanoyl hydroxyproline compounds preferably comprise from about 0.01% to about 10%, more preferably from about 0.1% to about 5%, even more preferably from about 0.1% to about 2% by weight of the composition Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably, the dipalmitoyl hydroxyproline is the triethanolamine salt of dipalmitoyl hydroxyproline.

7. Flavonoids

The compositions of the present invention may comprise a flavonoid compound. Flavonoids are broadly disclosed in U.S. Pat. Nos. 5,686,082 and 5,686,367. Examples of flavonoids particularly suitable for use in the present invention are one or more flavones, one or more isoflavones, one or more coumarins, one or more chromones, one or more dicoumarols, one or more chromanones, one or more chromanols, isomers (e.g., cis/trans isomers) thereof, and mixtures thereof.

Preferred for use herein are flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof.

Flavonoid compounds useful herein are commercially available from a number of sources, e.g., Indofine Chemical Company, Inc., Steraloids, Inc., and Aldrich Chemical Company, Inc.

The herein described flavonoid compounds preferably comprise from about 0.01% to about 20%, more preferably from about 0.1% to about 10%, and even more preferably from about 0.5% to about 5%, by weight of the composition.

8. N-acyl Amino Acid Compound

The topical compositions of the present invention may comprise a safe and effective amount of one or more N-acyl amino acid compounds. The amino acid can be one of any of the amino acids known in the art. The N-acyl amino acid compounds of the present invention correspond to the formula: 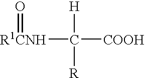
wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W.H. Freeman and Company. R¹ can be C₁ to C₃₀, saturated or unsaturated, straight or branched, substituted or unsubstituted alkyls; substituted or unsubstituted aromatic groups; or mixtures thereof.

Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula: 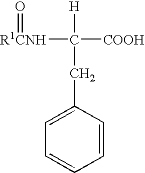
wherein R¹ can be C₁ to C₃₀, saturated or unsaturated, straight or branched, substituted or unsubstituted alkyls; substituted or unsubstituted aromatic groups; or mixtures thereof.

N-acyl Tyrosine corresponds to the following formula: 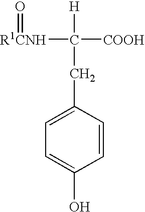
wherein R¹ can be C₁ to C₃₀, saturated or unsaturated, straight or branched, substituted or unsubstituted alkyls; substituted or unsubstituted aromatic groups; or mixtures thereof.

Particularly useful as a topical skin tone evening cosmetic agent is N-undecylenoyl-L-phenylalanine. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

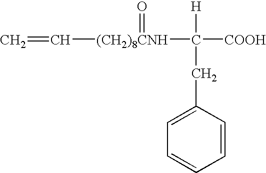

As used herein, N-undecylenoyl-L-phenylalanine is commercially available under the tradename Sepiwhite® from SEPPIC.

In the composition of the present invention, the N-acyl amino acid preferably comprises from about 0.0001% to about 25%, more preferably from about 0.001% to about 10%, more preferably from about 0.01% to about 5%, and even more preferably from about 0.02% to about 2.5% by weight of the composition.

9. Retinoid

The compositions of this invention may contain a safe and effective amount of a retinoid, such that the resultant composition is safe and effective for regulating keratinous tissue condition, preferably for regulating visible and/or tactile discontinuities in skin, more preferably for regulating signs of skin aging. The compositions preferably contain from about 0.001% to about 10%, more preferably from about 0.005% to about 2%, even more preferably from about 0.008% to about 1%, still more preferably from about 0.01% to about 0.5%, by weight of the composition, of the retinoid. The optimum concentration used in a composition will depend on the specific retinoid selected since their potency does vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. Preferred retinoids are retinol, retinyl palmitate, retinyl acetate, retinyl propionate, retinal and combinations thereof. More preferred is retinyl propionate, used even more preferably from about 0.1% to about 0.3%.

10. Peptide

The compositions of the present invention may contain a safe and effective amount of a peptide, including but not limited to, di-, tri-, tetra-, penta-, and hexa-peptides and derivatives thereof. The compositions contain preferably from about 1×10⁻⁷% to about 20%, more preferably from about 1×10⁻⁶% to about 10%, even more preferably from about 1×10⁻⁵% to about 5%, by weight of the composition.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as Iamin). A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmnitoyl-lys-thr-thr-lys-ser and is commercially available from Sederma.

11. Water-Soluble Vitamins

The compositions of the present invention may contain a safe and effective amount of one or more water-soluble vitamins. Examples of water-soluble vitamins include, but are not limited to, water-soluble versions of vitamin B, vitamin B derivatives, vitamin C, vitamin C derivatives, vitamin K, vitamin K derivatives, vitamin D, vitamin D derivatives, vitamin E, vitamin E derivatives, provitamins thereof, such as panthenol and mixtures thereof. When vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the vitamin compound.

12. Particulate Material

The compositions of the present invention may contain one or more particulate materials. Nonlimiting examples of particulate materials useful in the present invention include colored and uncolored pigments, interference pigments, inorganic powders, organic powders, composite powders, optical brightener particles, and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. Preferably, particulate materials are present in the composition in levels of from about 0.01% to about 20%, more preferably from about 0.05% to about 10%, still more preferably from about 0.1% to about 5%, by weight of the composition. There are no specific limitations as to the pigment, colorant or filler powders used in the composition.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, aluminum oxide, silicone resin, barium sulfate, calcium carbonate, cellulose acetate, PTFE, polymethyl methacrylate, starch, modified starches such as aluminun starch octenyl succinate, silk, glass, and mixtures thereof. Preferred organic powders/fillers include, but are not limited, to polymeric particles chosen from the methylsilsesquioxane resin microspheres such as for example those sold by Toshiba silicone under the name Tospearl 145A; microspheres of polymethylmethacrylates such as those sold by Seppic under the name Micropearl M 100; the spherical particles of crosslinked polydimethylsiloxanes, especially such as those sold by Dow Corning Toray Silicone under the name Trefil E 506C or Trefil E 505C, sphericle particles of polyamide and more specifically Nylon 12, especially such as those sold by Atochem under the name Orgasol 2002D Nat CO₅, polystyerene microspheres such as for example those sold by Dyno Particles under the name Dynospheres, ethylene acrylate copolymer sold by Kobo under the name FloBead EA209, PThE, polypropylene, aluminium starch ocetenylsuccinate such as those sold by National Starch under the name Dry Flo, microspheres of polyethylene such as those sold by Equistar under the name of Microthene FN510-00, silicone resin, polymethylsilsesquioxane silicone polymer, platelet shaped powder made from L-lauroyl lysine, and mixtures thereof. Especially preferred are spherical powders with an average primary particle size of from about 0.1 to about 75 microns, preferably from about 0.2 to about 30 microns.

Also useful herein are interference pigments. Interference pigments, for purposes of the present specification are defined as thin platelike layered particles having two or more layers of controlled thickness with different refractive indices that yield a characteristic reflected color from the interference of typically two, but occasionally more, light reflections, form different layers of the platelike particle. The most common examples of interference pigments are micas layered with about 50-300 nm films of TiO2, Fe2O3, silica, tin oxide, and/or Cr203. Such pigments are often peralescent. Pearl pigments reflect, refract and transmit light because of the transparency of pigment particles and the large difference in the refractive index of mica platelets and, for example, the titanium dioxide coating. Useful intereference pigments are available commercially from a wide variety of suppliers, for example, Rona (Timiron™ and Dichrona™), Presperse (Flonac™), Englehard (Duochrome™), Kobo (SK45-R and SK45-G), BASF (Sicopearls) and Eckart (e.g. Prestige Silk Red). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887.

Preferred colored or uncolored non-interference-type pigments have a primary average particle size of from about 10 nm to about 100,000 nm, more preferably from about 15 nm to about 5,000 nm, even more preferably from about 20 nm to about 1000 nm. Mixtures of the same or different pigment/powder having different particle sizes are also useful herein (e.g., incorporating a TiO2 having a primary particle size of from about 100 nm to about 400 nm with a TiO2 having a primary particle size of from about 10 nm to about 50 nm).

The pigments/powders of the current invention can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobic treatments being preferred. Particularly useful hydrophobic pigment treatments include polysiloxane treatments such as those disclosed in U.S. Pat. No. 5,143,722.

13. Sunscreen Actives

The compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Particularly suitable sunscreen agents are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Preferred organic sunscreen actives useful in the compositions of the present invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

A safe and effective amount of the sunscreen active is used, typically from about 1% to about 20%, more typically from about 2% to about 10% by weight of the composition. Exact amounts will vary depending upon the sunscreen chosen and the desired Sun Protection Factor (SPF).

14. Anti-Cellulite Agents

The compositions of the present invention may also comprise a safe and effective amount of an anti-cellulite agent. Suitable agents may include, but are not limited to, xanthine compounds (e.g., caffeine, theophylline, theobromine, and aminophylline).

15. Butylated Hydroxytoluene (BHT) and Butylated Hydroxyanisole (BHA)

The topical compositions of the present invention may comprise a safe and effective amount of BHT or BHA. The BHT useful herein can be described by the general structure: 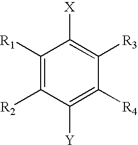
wherein X is OH or SH;
Y is selected from the group consisting of H, OH, OR₅, COOR₅, alkyl, cycloalkyl, heteroalkyl, heterocycloalkyl, aromatic, heteroaromatic, carboxarido, sulfonamido, carbamate, urea, and trialkylsilyl;
R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen;
R₅ is selected from the group consisting of alkyl, cycloalkyl, heteroalkyl, heterocycloalkyl, aromatic, heteroaromatic, trialkylsilyl, acyl, and hydrogen.

In the composition of the present invention, BHT or BHA preferably comprises from about 0.0001% to about 20% by weight of the composition, more preferably from about 0.001% to about 10%, more preferably from about 0.01% to about 5%, and even more preferably from about 0.1% to about 0.5%.

3. Dermatologically Acceptable Carrier

The topical compositions of the present invention also comprise a dermatologically acceptable carrier for the composition. The phrase “dermatologically acceptable carrier”, as used herein, means that the carrier is suitable for topical application to the keratinous tissue, has good aesthetic properties, is compatible with the actives of the present invention and any other components, and will not cause any safety or toxicity concerns. A safe and effective amount of carrier is from about 50% to about 99.99%, preferably from about 60% to about 99.9%, more preferably from about 70% to about 98%, and even more preferably from about 80% to about 95% of the composition.

The carrier can be in a wide variety of forms. Non-limiting examples include simple solutions (water or oil based), emulsions and solid forms (gels, sticks). For example, emulsion carriers, including, but not limited to, oil-in-water, water-in-oil, water-in-oil-in-water, and oil-in-water-in-silicone emulsions, are useful herein.

Preferred carriers comprise an emulsion such as oil-in-water emulsions (e.g., silicone in water) and water-in-oil emulsions, (e.g., water-in-silicone emulsions). As will be understood by the skilled artisan, a given component will distribute primarily into either the water or oil phase, depending on the water solubility/dispensability of the component in the composition. Oil-in-water emulsions are especially preferred.

Emulsions according to the present invention generally contain an aqueous phase and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

4. Optional Components

The compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

1. Desquamation Actives

A safe and effective amount of a desquamation active may be added to the compositions of the present invention, more preferably from about 0.01% to about 10%, even more preferably from about 0.1% to about 5%, also preferably from about 0.5% to about 2%, by weight of the composition. One desquamation system that is suitable for use herein comprises salicylic acid and zwitterionic surfactants and is described in U.S. Pat. No. 5,652,228. Zwitterionic surfactants such as described in these applications are also useful as desquamatory agents herein, with cetyl betaine being particularly preferred.

2. Anti-Acne Actives

The compositions of the present invention may comprise a safe and effective amount of one or more anti-acne actives. Examples of useful anti-acne actives include resorcinol, sulfur, erythromycin, zinc, etc. Further examples of suitable anti-acne actives are described in further detail in U.S. Pat. No. 5,607,980.

3. Anti-Wrinkle Actives/Anti-Atrophy Actives

The compositions of the present invention may further comprise a safe and effective amount of one or more anti-wrinkle actives or anti-atrophy actives. Exemplary anti-wrinkle/anti-atrophy actives suitable for use in the compositions of the present invention include hydroxy acids (e.g., glycolic acid), keto acids (e.g., pyruvic acid), phytic acid, lysophosphatidic acid, stilbenes, cinnamates, resveratrol, kinetin, zeatin, dimethylaminoethanol, peptides from natural sources (e.g., soy peptides), and salts of sugar acids (e.g., Mn gluconate).

4. Anti-Oxidants/Racial Scavengers

The compositions of the present invention may include a safe and effective amount of an anti-oxidant/radical scavenger. A safe and effective amount of an anti-oxidant/radical scavenger may be added to the compositions of the subject invention, preferably from about 0.01% to about 10%, more preferably from about 0.1% to about 5%, of the composition.

Anti-oxidants/radical scavengers such as tocopherol (vitamin E), tocopherol sorbate, tocopherol acetate, other esters of tocopherol, 6-hydroxy-2,5,7,8-tetramethylchroman-2-carboxylic acid (commercially available under the tradename Trolox^{R}), amines (e.g., N,N-diethylhydroxylamine, amino-guanidine), nordihydroguaiaretic acid, bioflavonoids, amino acids, silymarin, tea extracts, and grape skin/seed extracts may be used. Preferred anti-oxidants/radical scavengers are selected from esters of tocopherol, more preferably tocopherol acetate.

5. Conditioning Agents

The compositions of the present invention may contain a safe and effective amount of a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof. Also useful herein are the propoxylated glycerols described in U.S. Pat. No. 4,976,953.

Also useful are various C₁-C₃₀ monoesters and polyesters of sugars and related materials. These esters are derived from a sugar or polyol moiety and one or more carboxylic acid moieties.

Preferably, the conditioning agent is selected from urea, guanidine, sucrose polyester, panthenol, dexpanthenol, allantoin, glycerol, and combinations thereof.

6. Chelators

The compositions of the present invention may also comprise a safe and effective amount of a chelator or chelating agent. As used herein, “chelator” or “chelating agent” means an active agent capable of removing a metal ion from a system by forming a complex so that the metal ion cannot readily participate in or catalyze oxygen radical formation.

A safe and effective amount of a chelating agent may be added to the compositions of the subject invention, preferably from about 0.1% to about 10%, more preferably from about 1% to about 5%, by weight of the composition. Exemplary chelators that are useful herein are disclosed in U.S. Pat. No. 5,487,884. Preferred chelators useful in compositions of the subject invention are furildioxime and derivatives thereof.

7. Anti-Inflammatory Agents

A safe and effective amount of an anti-inflammatory agent may be added to the compositions of the present invention, preferably from about 0.01% to about 10%, more preferably from about 0.5% to about 5%, by weight of the composition.

Steroidal anti-inflammatory agents, include but are not limited to, corticosteroids such as hydrocortisone. A second class of anti-inflammatory agents, which is useful in the compositions, includes the nonsteroidal anti-inflammatory agents. The varieties of compounds encompassed by this group are well known to those skilled in the art. Specific non-steroidal anti-inflammatory agents useful in the composition of the present invention include, but are not limited to, salicylates, flufenamic acid, etofenamate, aspirin, and mixtures thereof.

Additional anti-inflammatory agents useful herein include allantoin and compounds of the Licorice (the plant genus/species Glycyrrhiza glabra) family, including glycyrrhetic acid, glycyrrhizic acid, and derivatives thereof (e.g., salts and esters).

8. Tanning Actives

The compositions of the present invention may comprise a tanning active. When present, it is preferable that the compositions comprise from about 0.1% to about 20%, more preferably from about 2% to about 7%, and even more preferably from about 3% to about 6%, by weight of the composition, of a tanning active. A preferred tanning active is dihydroxyacetone.

9. Skin Lightening Agents

The compositions of the present invention may comprise a skin lightening agent. When used, the compositions preferably comprise from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, and tranexamic acid. Other skin lightening materials suitable for use herein include Acitwhite® (Cognis), Emblica® (Rona), Azeloglicina (Sinerga) and extracts (e.g. mulberry extract).

10. Antimicrobial and Antifungal Actives

The compositions of the present invention may comprise an antimicrobial or antifungal active. A safe and effective amount of an antimicrobial or antifungal active may be added to the present compositions, preferably, from about 0.001% to about 10%, more preferably from about 0.01% to about 5%, and even more preferably from about 0.05% to about 2% by weight of the composition.

Preferred examples of actives useful herein include those selected from the group consisting of benzoyl peroxide, 3-hydroxy benzoic acid, glycolic acid, lactic acid, 4-hydroxy benzoic acid, 2-hydroxybutanoic acid, 2-hydroxypentanoic acid, 2-hydroxyhexanoic acid, phytic acid, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, ciclopirox, lidocaine hydrochloride, clotrimazole, miconazole, ketoconazole, neocycin sulfate, and mixtures thereof

11. Thickening Agents (Including Thickeners and Gelling Agents)

The compositions of the present invention can comprise one or more thickening agents, preferably from about 0.05% to about 10%, more preferably from about 0.1% to about 5%, and even more preferably from about 0.25% to about 4%, by weight of the composition.

Nonlimiting classes of thickening agents include those selected from the group consisting of:

a. Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀-₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B.F. Goodrich. Examples of carboxylic acid polymer thickeners useful herein are those selected from the group consisting of carbomers, acrylates/C₁₀-C₃₀ alkyl acrylate crosspolymers, and mixtures thereof.

b. Crosslinked Polyacrylate Polymers

The compositions of the present invention can optionally comprise crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. Nos. 5,100,660; 4,849,484; 4,835,206; 4,628,078; 4,599,379, and EP 228,868.

c. Polyacrylamide Polymers

The compositions of the present invention can optionally comprise polyacrylamide polymers, especially nonionic polyacrylamide polymers including substituted branched or unbranched polymers. Preferred among these polyacrylamide polymers is the nonionic polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

d. Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogell™ CS 11 from Michel Mercier Products Inc.

e. Gums

Other thickening and gelling agents useful herein include materials that are primarily derived from natural sources. Nonlimiting examples of these gelling agent gums include materials selected from the group consisting of acacia, agar, algin, alginic acid, ammonium alginate, amylopectin, calcium alginate, calcium carrageenan, carnitine, carrageenan, dextrin, gelatin, gellan gum, guar gum, guar hydroxypropyltimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

### Composition Forms

The topical compositions of the subject invention include, but are not limited to, lotions, milks, mousses, serums, sprays, aerosols, foams, sticks, pencils, gels, creams and ointments. Such compositions preferably contain from about 2% to about 50% of an emollient. As used herein, “emollient” refers to a material useful for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), contains numerous examples of materials suitable as an emollient. A preferred emollient is glycerin. Glycerin is preferably used in an amount of from about 0.001% to about 20%, more preferably from about 0.01% to about 15%, and even more preferably from about 0.1% to about 10%, by weight of the composition.

Compositions of this invention useful for cleansing (“cleansers”) are formulated with a suitable carrier (e.g., as described above, and from about 1% to about 90%, by weight of the composition, of a dermatologically acceptable surfactant).

The physical form of the cleansing compositions is not critical. The compositions can be, for example, formulated as toilet bars, liquids, shampoos, bath gels, hair conditioners, hair tonics, pastes, or mousses. Toilet bars are preferred since this is the form of cleansing agent most commonly used to wash the skin. Rinse-off cleansing compositions, such as shampoos, require a delivery system adequate to deposit sufficient levels of actives on the skin and scalp. A preferred delivery system involves the use of insoluble complexes. For a more complete disclosure of such delivery systems, see U.S. Pat. No. 4,835,148.

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

### Composition Preparation

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Methods for Regulating Keratinous Tissue Condition

The compositions of the present invention are useful for regulating a number of mammalian keratinous tissue conditions. Such regulation of keratinous tissue conditions includes prophylactic and therapeutic regulation. More specifically, such regulating methods are directed to, but are not limited to, preventing, retarding, and/or treating uneven skin tone, reducing the size of pores in mammalian skin, regulating oily/shiny appearance of mammalian skin, thickening keratinous tissue (i.e., building the epidermis and/or dermis and/or subcutaneous layers of the skin and where applicable the keratinous layers of the nail and hair shaft), preventing, retarding, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of mammalian skin, desquamating, exfoliating, and/or increasing turnover in mammalian skin, preventing, retarding, and/or treating hyperpigmentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin.

Regulating keratinous tissue conditions involves topically applying to the keratinous tissue a safe and effective amount of a composition of the present invention. The amount of the composition that is applied, the frequency of application and the period of use will vary widely depending upon the level of components of a given composition and the level of regulation desired, e.g., in light of the level of keratinous tissue damage present or expected to occur.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application are, in mg composition/cm² skin, from about 0.1 mg/cm² to about 20 mg/cm². A particularly useful application amount is about 0.5 mg/cm² to about 10 mg/cm².

Regulating keratinous tissue condition is preferably practiced by applying a composition in the form of a skin lotion, clear lotion, milky lotion, cream, gel, foam, ointment, paste, emulsion, spray, conditioner, tonic, cosmetic, lipstick, foundation, nail polish, after-shave, or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, fingernails, toenails, scalp hair, eyelashes, eyebrows, etc.) The application of the present compositions may be done using the palms of the hands and/or fingers or an implement (e.g., a cotton ball, swab, pad, etc.)

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, and the like). The patch can be occlusive, semi-occlusive or non-occlusive. The composition can be contained within the patch or be applied to the skin prior to application of the patch. The patch can also include additional actives such as chemical initiators for exothermic reactions such as those described in PCT application WO 9701313. The patch can also contain a source of electrical energy (e.g., a battery) to, for example, increase delivery of the composition and active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-5

### Moisturizing Oil-In-Water Lotions/Creams

| | | | | |
|---|---|---|---|---|
| 1 | 2 | 3 | 4 | 5 |

Water Phase:

| | | | | | |
|---|---|---|---|---|---|
| Water | qs | qs | qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | 0.5 | 0.1 | 0.5 | 0.1 | 0.5 |
| Benzyl alcohol | 0.25 | 0.25 | 0.25 | 0.25 | 0.25 |
| GLW75CAP-MP (75% aq. | — | 0.5 | 0.5 | — | — |
| TiO2 dispersion)¹ | | | | | |
| Hexamidine diisethionate | — | 0.1 | — | — | — |
| Palmitoyl-pentapeptide² | 0.0003 | — | 0.0001 | — | 0.0003 |
| N-acetyl glucosamine | 2 | 1 | 2 | 2 | 1 |
| Soy Isoflavone | 0.5 | — | — | — | — |

Oil Phase:

| | | | | | |
|---|---|---|---|---|---|
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Dipalmitoylhydroxyproline | — | — | — | 1.0 | — |
| Undecylenoyl Phenylalanine | — | 0.5 | — | — | — |
| Phytosterol | — | — | 0.5 | — | 1.0 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |

Thickener:

| | | | | | |
|---|---|---|---|---|---|
| Polyacrylamide/C13-14 | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isohexadecane/ | | | | | |
| polysorbate 80 | | | | | |

Additional Ingredients:

| | | | | | |
|---|---|---|---|---|---|
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

¹Available from Kobo products
²Palmitoyl-lysine-threonine-threonine-lysine-serine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (eg., with a Tekmar T-25). Then, add the thickener to the emulsion and cool the emulsion to 45° C. while stirring. At 45° C., add the remaining ingredients. Cool the product and stir to 30° C. and pour into suitable containers.

### Examples 6-11

### Moisturizing Silicone-In-Water Serums/Lotions

| | | | | | |
|---|---|---|---|---|---|
| 6 | 7 | 8 | 9 | 10 | 11 |

Water Phase:

| | | | | | | |
|---|---|---|---|---|---|---|
| Water | qs | qs | qs | qs | qs | qs |
| Glycerin | 3 | 5 | 7 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| Sodium | 0.5 | 0.1 | — | 0.1 | 0.5 | 0.1 |
| Dehydroacetate | | | | | | |
| D-panthenol | 0.5 | 0.1 | | 0.5 | 1.5 | 0.5 |
| GLW75CAP-MP | — | 0.4 | — | — | — | 0.4 |
| (75% aq. TiO2 | | | | | | |
| dispersion)¹ | | | | | | |
| Ascorbyl Glucoside | — | — | — | — | — | 1 |
| Palmitoyl | — | — | — | — | 0.0003 | — |
| pentapeptide² | | | | | | |
| Soy Isoflavone | — | 1 | — | — | — | — |
| N-acetyl glucosamine | 2 | — | 2 | — | 5 | — |

Silicone/Oil Phase:

| | | | | | | |
|---|---|---|---|---|---|---|
| Cyclomethicone D5 | 10 | 5 | 5 | 10 | 7.5 | 10 |
| Dow Corning 9040 | — | 10 | 5 | 5 | 7.5 | 5 |
| silicone elastomer³ | | | | | | |
| KSG-15AP silicone | 5 | — | 5 | 5 | 7.5 | 5 |
| Elastomer⁴ | | | | | | |
| Dimethione/ | — | 2 | 2 | 1 | 2 | 1 |
| dimethiconol | | | | | | |
| Dimethicone 50 csk | 1 | — | — | — | — | — |
| Salicylic Acid | — | — | 1.5 | — | — | — |
| Phytosterol | — | — | — | 1.0 | — | 0.1 |
| PPG-15 Stearyl Ether | — | — | 4 | 4 | — | — |
| Dehydroacetic acid | — | — | 0.5 | — | — | — |
| Undecylenoyl | — | — | 0.5 | — | — | — |
| Phenylalanine | | | | | | |
| BHT | — | 0.5 | — | — | — | — |
| Vitamin E Acetate | — | 0.5 | 0.1 | 0.1 | — | 0.1 |

Thickener:

| | | | | | | |
|---|---|---|---|---|---|---|
| Polyacrylamide/C13-14 | 2.5 | 2.5 | — | — | — | 3 |
| isoparaffin/laureth-7 | | | | | | |
| Sodiumacrylate/ | — | — | — | 3 | — | — |
| sodium acryloyl | | | | | | |
| dimethyl taurate | | | | | | |
| copolymer/isohexadecane/ | | | | | | |
| polysorbate 80 | | | | | | |
| Acrylates/C10-30 | — | — | 0.6 | — | 0.5 | — |
| alkyl acrylates | | | | | | |
| crosspolymer | | | | | | |

Undecylenoyl Phenylalanine Premix

| | | | | | | |
|---|---|---|---|---|---|---|
| Undecylenoyl | — | — | — | — | 1 | — |
| Phenylalanine | | | | | | |
| Water | — | — | — | — | 24 | — |
| Triethanolamine | — | — | — | — | 0.5 | — |

Dipalmitoyl Hydroxy-Proline Premix:

| | | | | | | |
|---|---|---|---|---|---|---|
| Water | — | — | — | — | — | 4.4 |
| Triethanolamine | — | — | — | — | — | 0.1 |
| Dipalmitoylhyroxyproline | — | — | — | — | — | 1.0 |

Additional Ingredients:

| | | | | | | |
|---|---|---|---|---|---|---|
| Triethanolamine | — | — | — | — | 0.6 | — |
| Polymethylsilsequioxane | 0.5 | 0.5 | 1.0 | 1 | 1 | 0.5 |
| Polyethylene | — | 0.5 | 0.5 | 1.0 | — | — |
| Flamenco Summit | — | — | 1.0 | — | — | — |
| Green G30D⁵ | | | | | | |
| Silca | — | — | 1 | 0.5 | — | — |
| Prestige Silk Red⁶ | — | — | — | 1.0 | 1.0 | 1.0 |

¹GLW75CAP-MP, 75% aqueous titanium dioxide dispersion from Kobo
²Palmitoyl-lysine-threonine-threonine-lysine-serine available from Sederma
³A silicone elastomer dispersion from Dow Corning Corp
⁴A silicone elastomer dispersion from Shin Etsu,
⁵Titanium dioxide and tin oxide coated mica green interference pigment from Engelhard
⁶Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the dipalmitoyl hydroxyproline premix and/or undecylenoyl phenylalanine premix by combining the premix ingredients in a suitable container, heat to about 70° C. while stirring, and cool to room temperature while stirring. Add half the thickener and then the silicone/oil phase to the water phase and mill the resulting emulsion (eg., with a Tekmar T-25). Add the remainder of the thickener, the dipalmitoyl hydroxyproline premix and/or undecylenoyl phenylalanine premix, and then the remaining ingredients to the emulsion while stirring. Once the composition is uniform, pour the product into suitable containers.

### Examples 12-17

### Moisturizing Water-In-Silicone Creams/Lotions

| | | | | | | |
|---|---|---|---|---|---|---|
| Component | 12 | 13 | 14 | 15 | 16 | 17 |

Phase A

| | | | | | | |
|---|---|---|---|---|---|---|
| water | qs | qs | qs | qs | qs | qs |
| allantoin | 0.2 | 0.2 | 0.2 | 0.2 | 0.2 | 0.2 |
| disodium EDTA | 0.1 | 0.1 | 0.1 | 0.1 | 0.1 | 0.1 |
| ethyl paraben | 0.2 | 0.2 | 0.2 | 0.2 | 0.2 | 0.2 |
| propyl paraben | 0.1 | 0.1 | 0.1 | 0.1 | 0.1 | 0.1 |
| Caffeine | — | 1 | — | — | — | 1 |
| BHT | — | 0.1 | — | 0.015 | — | — |
| dexpanthenol | 1 | 0.5 | 1 | 1 | 1 | 1 |
| glycerin | 7.5 | 10 | 15 | 7.5 | 5 | 15 |
| hexamidine isethionate | — | — | 0.1 | 0.5 | — | — |
| niacinamide | 2 | — | — | 2 | 3.5 | 5 |
| palmitoyl-pentapeptide¹ | — | — | — | — | 0.0003 | — |
| Phenylbenzimidazole sulfonic | — | — | — | — | 1 | — |
| acid | | | | | | |
| Sodium Dehydroacetate | 0.5 | — | — | 0.1 | 0.5 | 0.5 |
| benzyl alcohol | 0.25 | 0.25 | 0.25 | 0.25 | 0.25 | 0.25 |
| triethanolamine | — | — | — | — | 0.6 | — |
| green tea extract | 1 | 1 | 1 | 1 | 1 | 1 |
| Soy Isoflavone | — | 0.5 | — | — | — | — |
| N-acetyl glucosamine | 5 | — | 2 | 5 | 2 | — |
| sodium metabisulfite | 0.1 | 0.1 | 0.1 | 0.1 | 0.1 | 0.1 |

Phase B

| | | | | | | |
|---|---|---|---|---|---|---|
| cyclopentasiloxane | 15 | 15 | 18 | 15 | 15 | 18 |
| titanium dioxide | 0.5 | 0.5 | 0.75 | 0.5 | 0.5 | 0.75 |

Phase C

| | | | | | | |
|---|---|---|---|---|---|---|
| C12-C15 alkyl benzoate | — | — | — | 1.5 | 1.5 | — |
| vitamin E acetate | 0.5 | — | 1 | 0.5 | 0.5 | 1 |
| retinyl propionate | 0.3 | — | — | 0.2 | 0.2 | — |
| Undecylenoyl Phenylalanine | — | — | 0.5 | — | — | — |
| Dipalmitoyl hydroxyproline | — | 1 | — | — | — | — |
| Salicylic Acid | — | 1.5 | 1.5 | — | — | — |
| PPG-15 Stearyl Ether | 4 | 4 | 4 | — | — | — |
| Dehydroacetic Acid | — | 0.5 | 0.1 | — | — | — |
| phytosterol | 1 | 0.5 | — | — | — | — |

Phase D

| | | | | | | |
|---|---|---|---|---|---|---|
| KSG-21 silicone elastomer² | 4 | 4 | 5 | 4 | 4 | 5 |
| Dow Corning 9040 silicone | 15 | 15 | 12 | 15 | 15 | 12 |
| elastomer³ | | | | | | |
| Abil EM-97 Dimethicone | 0.5 | — | — | 0.5 | 0.5 | — |
| Copolyol⁴ | | | | | | |
| polymethylsilsesquioxane | 2.5 | 2.5 | 2 | 2.5 | 2.5 | 2 |
| Undecylenoyl Phenylalanine Premix | | | | | | |
| Undecylenoyl Phenylalanine | — | — | — | — | 1 | — |
| Water | — | — | — | — | 24 | — |
| Triethanolamine | — | — | — | — | 0.5 | — |

Phase E

| | | | | | | |
|---|---|---|---|---|---|---|
| Water | 8.8 | — | — | — | — | 8.85 |
| Triethanolamine | 0.2 | — | — | — | — | 0.25 |
| Dipalmitoylhyroxyproline | 0.5 | — | — | — | — | 1 |

¹Palmitoyl-lysine-threonine-threonine-lysine-serine available from Sederma
²KSG-21 is an emulsifying silicone elastomer available from Shin Etsu
³A silicone elastomer dispersion from Dow Corning Corp
⁴Abil EM-97 available from Goldschmidt Chemical Corporation

In a suitable vessel, blend the Phase A components together with a suitable mixer (e.g., Tekmar model RW20DZM) and mix until all of the components are dissolved. Then, blend the Phase B components together in a suitable vessel and mill using a suitable mill (e.g., Tekmar RW-20) for about 5 minutes. Add the Phase C components to the Phase B mixture with mixing. Then, add the Phase D components to the mixture of Phases B and C and then mix the resulting combination of Phase B, C and D components using a suitable mixer (e.g., Tekmar RW-20) for about 1 hour. If applicable, prepare the undecylenoyl phenylalanine premix and/or Phase E by combining all ingredients, heating the ingredients to 70° C. while stirring, and cooling back to room temperature while stirring. Add the undecylenoyl phenylalanine premix and/or Phase E to Phase A while mixing. Next, slowly add Phase A to the mixture of Phases B, C and D with mixing. Mix the resulting mixture continually until the product is uniform. Mill the resulting product for about 5 minutes using an appropriate mill (e.g., Tekmar T-25).

### Examples 18-22

### Oil in Water Mousse

| | | | | |
|---|---|---|---|---|
| 18 | 19 | 20 | 21 | 22 |

Water Phase:

| | | | | | |
|---|---|---|---|---|---|
| Water | qs | qs | qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | 0.5 | 0.1 | 0.5 | 0.1 | 0.5 |
| Benzyl alcohol | 0.25 | 0.25 | 0.25 | 0.25 | 0.25 |
| GLW75CAP-MP (75% aq. | — | 0.5 | 0.5 | — | — |
| TiO2 dispersion)¹ | | | | | |
| Undecylenoyl | 1 | — | 0.5 | — | — |
| Phenylalanine | | | | | |
| Hexamidine diisethionate | — | 0.1 | — | — | — |
| Palmitoyl-pentapeptide² | 0.0003 | — | 0.0001 | — | 0.0003 |
| N-acetyl glucosamine | 2 | 1 | 2 | 2 | 1 |
| Soy Isoflavone | 0.5 | — | — | — | — |

Oil Phase:

| | | | | | |
|---|---|---|---|---|---|
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Undecylenoyl | — | 0.5 | — | — | — |
| Phenylalanine | | | | | |
| Dipalmitoylhyroxyproline | — | — | — | 1.0 | — |
| Phytosterol | — | — | 0.5 | — | 1.0 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |

Thickener:

| | | | | | |
|---|---|---|---|---|---|
| Polyacrylamide/C13-14 | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isohexadecane/ | | | | | |
| polysorbate 80 | | | | | |

Additional Ingredients:

| | | | | | |
|---|---|---|---|---|---|
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

Propellant Phase

| | | | | | |
|---|---|---|---|---|---|
| 152 A HFC Propellant | 3 | 4 | 2 | 3 | 2 |
| A-70 Propellant | 3 | 2 | 4 | 3 | 4 |

¹Available from Kobo products
²Palmitoyl-lysine-threonine-threonine-lysine-serine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (eg., with a Tekmar T-25). Add the thickener to the emulsion and cool the emulsion to 45° C. while stirring. At 45° C., add the remaining ingredients. Cool the product with stirring to 30° C. and pour into suitable containers. Add propellant and product to a suitable aerosol container, and seal the container.

### Examples 23-28

### Silicone in Water Mousse

| | | | | | |
|---|---|---|---|---|---|
| 23 | 24 | 25 | 26 | 27 | 28 |

Water Phase:

| | | | | | | |
|---|---|---|---|---|---|---|
| Water | qs | qs | qs | qs | qs | qs |
| Glycerin | 3 | 5 | 7 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| Sodium Dehydroacetate | 0.5 | 0.1 | — | 0.1 | 0.5 | 0.1 |
| D-panthenol | 0.5 | 0.1 | — | 0.5 | 1.5 | 0.5 |
| GLW75CAP-MP (75% | — | 0.4 | — | — | — | 0.4 |
| aq. TiO2 dispersion)¹ | | | | | | |
| Ascorbyl Glucoside | — | — | — | — | — | 1 |
| Palmitoyl pentapeptide² | — | — | — | — | 0.0003 | — |
| Soy Isoflavone | — | 1 | — | — | — | — |
| N-acetyl glucosamine | 2 | — | 2 | — | 5 | — |

Silicone/Oil Phase:

| | | | | | | |
|---|---|---|---|---|---|---|
| Cyclomethicone D5 | 10 | 5 | 5 | 10 | 7.5 | 10 |
| Dow Corning 9040 | — | 10 | 5 | 5 | 7.5 | 5 |
| silicone elastomer³ | | | | | | |
| KSG-15AP silicone | 5 | — | 5 | 5 | 7.5 | 5 |
| Elastomer⁴ | | | | | | |
| Dimethione/ | — | 2 | 2 | 1 | 2 | 1 |
| dimethiconol | | | | | | |
| Dimethicone 50 csk | 1 | — | — | — | — | — |
| Salicylic Acid | — | — | 1.5 | — | — | — |
| Phytosterol | — | — | — | 1.0 | — | 0.1 |
| PPG-15 Stearyl Ether | — | — | 4 | 4 | — | — |
| Dehydroacetic acid | — | — | 0.5 | — | — | — |
| Undecylenoyl | — | — | 0.5 | — | — | — |
| Phenylalanine | | | | | | |
| BHT | — | 0.5 | — | — | — | — |
| Vitamin E Acetate | — | 0.5 | 0.1 | 0.1 | — | 0.1 |

Thickener:

| | | | | | | |
|---|---|---|---|---|---|---|
| Polyacrylamide/C13-14 | 2.5 | 2.5 | — | — | — | 3 |
| isoparaffin/laureth-7 | | | | | | |
| Sodiumacrylate/ | — | — | — | 3 | — | — |
| Sodium acryloyldimethyl | | | | | | |
| taurate | | | | | | |
| copolymer/isohexadecane/ | | | | | | |
| polysorbate 80 | | | | | | |
| Acrylates/C10-30 alkyl | — | — | 0.6 | — | 0.5 | — |
| acrylates crosspolymer | | | | | | |

Undecylenoyl Phenylalanine/Dipalmitoyl Hydroxyproline Premix

| | | | | | | |
|---|---|---|---|---|---|---|
| Undecylenoyl | — | — | — | — | 1 | — |
| Phenylalanine | | | | | | |
| Water | — | — | — | — | 24 | 9 |
| Triethanolamine | — | — | — | — | 0.5 | 0.2 |
| Dipalmitoylhyroxyproline | — | — | — | — | — | 1.0 |

Additional Ingredients:

| | | | | | | |
|---|---|---|---|---|---|---|
| Triethanolamine | — | — | — | — | 0.6 | — |
| Polymethyl | 0.5 | 0.5 | 1.0 | 1 | 1 | 0.5 |
| silsequioxane | | | | | | |
| Polyethylene | — | 0.5 | 0.5 | 1.0 | — | — |
| Flamenco Summit | — | — | 1.0 | — | — | — |
| Green G30D⁵ | | | | | | |
| Silca | — | — | 1 | 0.5 | — | — |
| Prestige Silk Red⁶ | — | — | — | 1.0 | 1.0 | 1.0 |

Propellant Phase

| | | | | | | |
|---|---|---|---|---|---|---|
| 152A HFCPropellant | 3 | 2 | 4 | 1 | 5 | 3 |
| A-70 Propellant | 3 | 4 | 2 | 5 | 1 | 3 |

¹GLW75CAP-MP, 75% aqueous titanium dioxide dispersion from Kobo
²Palmitoyl-lysine-threonine-threonine-lysine-serine available from Sederma
³A silicone elastomer dispersion from Dow Corning Corp
⁴A silicone elastomer dispersion from Shin Etsu,
⁵Titanium dioxide and tin oxide coated mica green interference pigment from Engelhard
⁶Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the undecylenoyl phenylalanine and/or dipalmitoyl hydroxyproline premix by combining the premix ingredients in a suitable container, heat to about 70° C. while stirring, and cool to room temperature while stirring. Add half the thickener and then the silicone/oil phase to the water phase and mill the resulting emulsion (eg., with a Tekmar T-25). Add the remainder of the thickener, the undecylenoyl phenylalanine and/or dipalmitoyl hydroxyproline premix, and then the remaining ingredients to the emulsion while stirring. Once the composition is uniform, pour the product into suitable containers. Add the product and propellant into an aerosol container. Seal the aerosol container.

### Examples 29-34

### Water Based Stick Formulations

| | | | | | |
|---|---|---|---|---|---|
| 29 | 30 | 31 | 32 | 33 | 34 |

Water Phase:

| | | | | | | |
|---|---|---|---|---|---|---|
| Water | qs | qs | qs | qs | qs | qs |
| Propylene Glycol | 15 | 25 | 20 | 15 | 25 | 20 |
| Dipropylene Glycol | 50 | 40 | 45 | 50 | 40 | 45 |
| Sodium Stearate | 6 | 6 | 6 | 6 | 6 | 6 |
| Triethanolamine | 0.2 | 0.25 | — | 0.7 | 0.6 | — |
| N-Acetyl-D-Glucosamine | — | 2.0 | 0.5 | — | — | 2.0 |
| Undecyenoyl Phenylalanine | — | 0.5 | — | 1 | — | — |
| Niacinamide | 2 | | 3.5 | | 2 | 3.5 |
| Sodium Dehydroacetate | 0.5 | 0.5 | 0.1 | 0.1 | 0.5 | 1.0 |
| Dipalmitoyl Hydroxyproline | 1 | — | — | 1 | 0.5 | — |

Combine all ingredients into an appropriate size container, heat to 85° C., cool and pour into stick containers at approximately 65° C.

### Examples 35-40

### Anhydrous Stick Formulations

| | | | | | |
|---|---|---|---|---|---|
| 35 | 36 | 37 | 38 | 39 | 40 |

Oil Phase:

| | | | | | | |
|---|---|---|---|---|---|---|
| Isopropyl Isostearate | 5 | 4 | 3 | 5 | 4 | 3 |
| Octylmethoxycinnamate | 5 | 2 | 2 | 5 | 2 | 2 |
| Cyclomethicone | qs | qs | qs | qs | qs | qs |
| Phenyl trimethicone | 5 | 5 | 5 | 5 | 5 | 5 |
| Stearyl Alcohol | 15 | 17 | 15 | 15 | 17 | 15 |
| Behenyl Alcohol | 1 | 1 | 1 | 1 | 1 | 1 |
| Undecylenoyl Phenylalanine | — | 0.5 | — | 1.0 | 0.5 | 0.5 |
| Dehydroacetic acid | 0.1 | 0.5 | 0.1 | 0.5 | 0.1 | 1.0 |
| Dipalmitoyl | 1 | — | 1.0 | — | 0.5 | — |
| Hydroxyproline | | | | | | |
| Phytosterol | 1 | 0.5 | — | — | 0.5 | 1 |
| Salicylic Acid | — | — | 0.5 | 1.5 | — | 1.0 |

Add all ingredients to an appropriate size container, heat to 75° C. then cool with stirring until mixture reaches approximately 45° C. Then pour the mixture into stick containers.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

1. A skin care composition comprising:
a) dehydroacetic acid, its isomers, salts, and derivatives thereof;
b) a safe and effective amount of at least two skin care actives selected from the group consisting of sugar amines; vitamin B3 compounds; phytosterols; salicylic acid compounds; hexamidines; dialkanoyl hydroxyproline compounds; flavonoids; n-acyl amino acid compounds; their derivatives, and combinations thereof; and
c) a dermatologically acceptable carrier for the dehydroacetic acid and the skin care actives.

2. The skin care composition of claim 1 wherein said dehydroacetic acid is present in an amount of from about 0.01% to about 10% by weight of the composition.

3. The skin care composition of claim 1 wherein said dehydroacetic acid is present in an amount of from about 0.05% to about 5% by weight of the composition.

4. The skin care composition of claim 1 wherein said dehydroacetic acid is present in an amount of from about 0.1% to about 1% by weight of the composition.

5. The skin care composition of claim 1 wherein said skin care actives are sugar amines and vitamin B3 compounds.

6. The skin care composition of claim 5 wherein said sugar amine is N-acetyl glucosamine.

7. The skin care composition of claim 5 wherein said vitamin B3 compound is niacinamide.

8. The skin care composition of claim 5 wherein said sugar amine is N-acetyl glucosamine and said vitamin B3 compound is niacinamide.

9. The skin care composition of claim 8 wherein said N-acetyl glucosamine is present in an amount of from about 0.5% to about 5% by weight of the composition and said niacinamide is present in an amount of from about 0.5% to about 5% by weight of the composition.

10. The skin care composition of claim 1 further comprising from about 0.001% to about 10%, by weight, of an additional component selected from the group consisting of desquamatory actives, anti-acne actives, wrinkle repair actives, anti-oxidants, radical scavengers, chelators, anti-inflammatory agents, tanning actives, skin lightening agents, antimicrobial actives, antifungal actives, thickening agents, and combinations thereof.

11. The skin care composition of claim 1 wherein said composition is an emulsion.

12. The skin care composition of claim 1 wherein said emulsion is selected from the group consisting of water-in-oil emulsions, oil-in-water emulsions, and combinations thereof.

13. The skin care composition of claim 12 wherein said oil-in-water emulsion a silicone-in-water emulsion.

14. A method of regulating the skin condition of mammalian keratinous tissue, said method comprising the step of topically applying to the skin of a mammal in need of such treatment the composition of claim 1.

15. A method of preventing, retarding, and/or treating uneven skin texture of a mammal, said method comprising the step of topically applying to the skin of a mammal needing such treatment the composition of claim 1.

16. A method of reducing the size of pores in mammalian skin, said method comprising the step of topically applying to the skin of a mammal in need of such treatment the composition of claim 1.

17. A method of regulating the oily/shiny appearance of mammalian skin, said method comprising the step of topically applying to the skin of a mammal in need of such treatment the composition of claim 1.

18. A skin care composition comprising:
a) dehydroacetic acid, its isomers, salts, and derivatives thereof;
b) a safe and effective amount of at least three skin care actives selected from the group consisting of sugar amines; vitamin B3 compounds; phytosterols; salicylic acid compounds; hexamidines; dialkanoyl hydroxyproline compounds; flavonoids; n-acyl amino acid compounds; retinoids; peptides; water-soluble vitamins; particulate materials; sunscreen actives; anti-cellulite agents; butylated hydroxytoluene and butylated hydroxyanisole; their derivatives, and combinations thereof; and
c) a dermatologically acceptable carrier for the dehydroacetic acid and the skin care actives.
